(19) United States
(12) Patent Application Publication
(10) Pub. No.: US 2025/0223271 A1
(43) Pub. Date: Jul. 10, 2025

(54) METHOD FOR CATALYTIC PREPARATION OF FURFURAL FROM BIOMASS USING PHENOLIC HYDROXYL-FUNCTIONALIZED COVALENT ORGANIC FRAMEWORK MATERIALS

(71) Applicant: QILU UNIVERSITY OF TECHNOLOGY (SHANDONG ACADEMY OF SCIENCES), Jinan, Shandong (CN)

(72) Inventors: Kai ZHANG, Jinan (CN); Peng GAN, Jinan (CN); Yuan JU, Jinan (CN); Baozhen GUO, Jinan (CN)

(73) Assignee: QILU UNIVERSITY OF TECHNOLOGY (SHANDONG ACADEMY OF SCIENCES), Jinan, Shandong (CN)

(21) Appl. No.: 18/689,499

(22) PCT Filed: Oct. 30, 2023
(86) PCT No.: PCT/CN2023/127953

§ 371 Date: Mar. 6, 2024

(30) Foreign Application Priority Data
Jun. 27, 2023 (CN) 202310770415.6

(51) Int. Cl. C07D 307/50; B01J 31/06; C08G 12/30

(52) U.S. Cl. CPC C07D 307/50; B01J 31/06; C08G 12/30

(57) ABSTRACT

A method for catalytic preparation of furfural from biomass using phenolic hydroxyl-functionalized covalent organic framework materials is provided. The present invention uses monomers containing phenolic hydroxyl groups, synthesizing imine-functionalized covalent organic framework materials (COFs) through Schiff base reactions and dehydration condensation. The constructed phenolic hydroxyl-functionalized COFs have a large specific surface area, rich pore structure, high crystallinity, strong thermal stability, and numerous acidic sites, enabling efficient catalysis of biomass raw materials to produce furfural. Innovatively, the present invention applies phenolic hydroxyl-functionalized COFs in the catalytic reaction for preparing furfural from biomass, opening a new sustainable path for the high-value utilization of renewable biomass resources to produce furfural chemicals. It allows for the high-selectivity production of furfural from biomass under mild conditions, with high stability, addressing issues such as poor selectivity and difficulty in reusing catalysts in the production process of furfural.

The present invention claims priority to the Chinese patent application submitted to the China National Intellectual Property Administration on Jun. 27, 2023, with the application number 202310770415.6, titled “method for catalytic preparation of furfural from biomass using phenolic hydroxyl-functionalized covalent organic framework materials”. The entire contents of this Chinese patent application are incorporated herein by reference and constitute a part of the present invention.

### TECHNICAL FIELD

The present invention belongs to the field of biomass catalytic conversion technology and biomass chemical engineering technology, specifically concerning a method for catalytic preparation of furfural from biomass using phenolic hydroxyl-functionalized covalent organic framework materials.

### BACKGROUND

Any discussion of the prior art throughout the specification should not be taken as an admission that such prior art is widely known or forms part of the common general knowledge in the art.

Biomass, as a renewable organic resource, has injected new vitality into the sustainable development of modern society. After pretreatment, biomass can be transformed into platform compounds, which in turn can derive a variety of value-added chemicals. Among them, furfural, due to its potential applications in the direction of biomass energy, is considered a multifunctional bio-based C5 platform molecule. The transformation of biomass to furfural mainly includes hydrolysis and dehydration steps, with acidic catalysts being the key to affecting these steps.

Currently, acidic catalysts used for the conversion of biomass to furfural include homogeneous and heterogeneous catalysts. Homogeneous catalysts, being in the same phase as the reactants, have high catalytic performance, but separation and recovery have always been a key issue restricting their development. Heterogeneous catalysts, having a distinct phase interface from the reactants, are easy to recover and reuse, but usually require external forces (such as mechanical stirring) to enhance contact with the reactants, and their catalytic performance is lower than that of homogeneous catalysts. Therefore, developing new types of heterogeneous catalysts with excellent catalytic performance is key to the rapid development of the furfural industry.

Covalent Organic Frameworks (COFs) materials are the best choice to meet the above needs. COFs material is a class of crystalline nanomaterials that can integrate organic units into well-defined two-dimensional and three-dimensional polymers, featuring high designability. However, there is currently no research in the industry on the use of covalent organic frameworks to catalyze the high-selectivity production of furfural from biomass under mild conditions.

### SUMMARY

To address the aforementioned issues, the present invention provides a method for catalytic preparation of furfural from biomass using phenolic hydroxyl-functionalized covalent organic framework materials. Specifically, biomass raw materials, phenolic hydroxyl-functionalized COFs material, and solvent are successively added to a reactor, filled with an inert gas, and reacted at a certain temperature to prepare furfural with high selectivity.

To achieve the above objectives, the present invention adopts the following technical solutions:

The first aspect of the present invention provides a method for catalytic preparation of furfural from biomass using phenolic hydroxyl-functionalized covalent organic framework materials, comprising:
using aldehyde or/and amino compounds containing phenolic hydroxyl functional groups as monomers, through solvothermal, microwave, or grinding methods, to initiate a Schiff base reaction followed by dehydration condensation to synthesize phenolic hydroxyl-functionalized covalent organic frameworks (COFs); and
mixing biomass raw materials and the phenolic hydroxyl-functionalized COFs in a solvent to achieve a uniform mixture, which is then subjected to catalytic reaction under an inert atmosphere at 160-210° C. for 0.5-5 hours to produce furfural.

The present invention introduces a method for catalytic preparation of furfural from biomass using phenolic hydroxyl-functionalized covalent organic framework materials. The rich porous structure of covalent organic frameworks provides ample diffusion space for the production of furfural. The phenolic hydroxyl-functionalized COFs of the present invention possess a large number and evenly distributed acidic sites, which can catalyze the high-selectivity production of furfural from biomass under mild conditions. The present invention opens a new pathway for the efficient production of biomass energy, addressing issues such as poor selectivity and difficulty in reusing catalysts in the furfural production process, among others.

The second aspect of the present invention provides a method for preparation of phenolic hydroxyl-functionalized covalent organic framework materials, comprising:
using 1,3,5-tri-(4-aminophenyl) triazine and 2,5-dihydroxyterephthalaldehyde as monomers, through solvothermal, microwave, or grinding methods, to initiate a Schiff base reaction followed by dehydration condensation to synthesize phenol hydroxyl-functionalized covalent organic frameworks (COFs).

The third aspect of the present invention provides an application of the aforementioned phenolic hydroxyl-functionalized covalent organic framework materials in the catalytic preparation of furfural from biomass.

The beneficial effects of the present invention include:

(1) the present invention proposes the application of COFs in the catalytic production of furfural, opening a new pathway for the efficient production of biomass energy.

(2) The present invention synthesizes COFs using monomers containing phenolic hydroxyl groups, which have a large number and evenly distributed acidic sites, enabling the high-selectivity production of furfural from biomass under mild conditions.

(3) The COFs prepared by the present invention are highly stable, can be reused multiple times with stable performance.

(4) The method of the present invention is simple and practical, easy to promote.

### BRIEF DESCRIPTION OF THE DRAWINGS

The accompanying drawings to the specification, which form part of the present invention, are used to provide a further understanding of the present invention, and the illustrative examples of the present invention and the description thereof are used to explain the present invention and are not unduly limiting the present invention.

FIG. 1 is a process flow diagram of present invention.

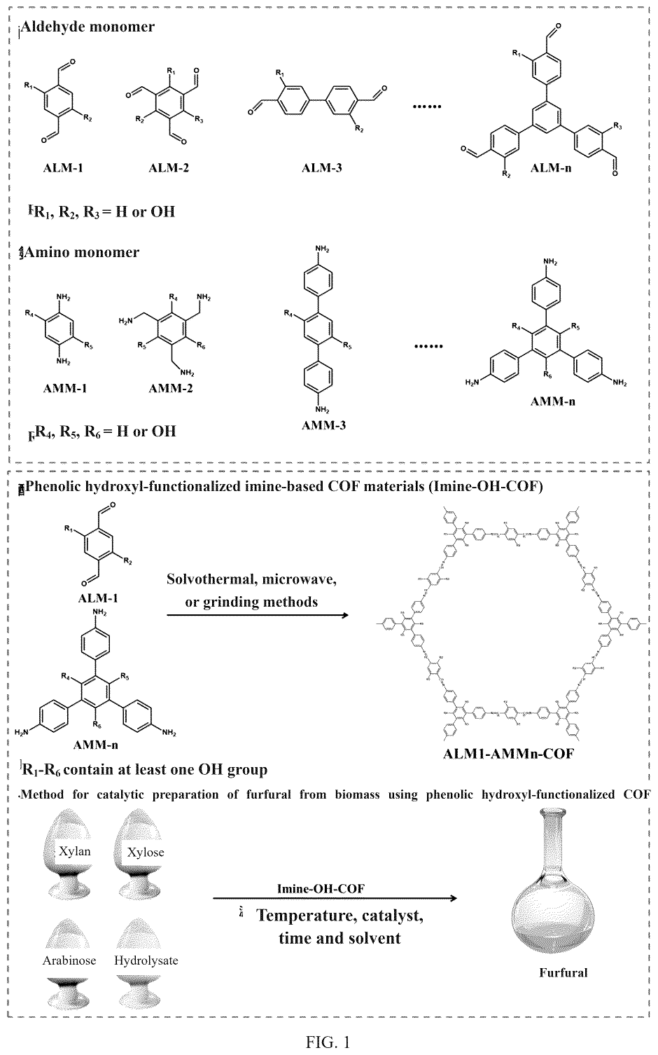

FIG. 2 shows an infrared and XRD spectra of the COFs prepared in the example of the present invention.

### DESCRIPTION OF THE PREFERRED EMBODIMENTS

It should be noted that the following detailed descriptions are all illustrative and intended to provide further clarification of the present invention. Unless otherwise specified, all technical and scientific terms used in the present invention have the same meaning as commonly understood by those of ordinary skill in the art to which the present invention belongs.

A method for catalytic preparation of furfural from biomass using phenolic hydroxyl-functionalized covalent organic framework materials, comprising:
using aldehyde or/and amino compounds containing phenolic hydroxyl functional groups as monomers, through solvothermal, microwave, or grinding methods, to initiate a Schiff base reaction followed by dehydration condensation to synthesize phenolic hydroxyl-functionalized COFs; and
mixing biomass raw materials and the phenolic hydroxyl-functionalized COFs in a solvent to achieve a uniform mixture, which is then subjected to catalytic reaction under an inert atmosphere at 160-210° C. for 0.5-5 hours to produce furfural.

In some embodiments, the biomass raw materials include, but are not limited to, xylan, xylose, arabinose, and biomass hydrolysate.

In some embodiments, the solvent is at least one of water, tetrahydrofuran, toluene, and γ-valerolactone.

In some embodiments, the inert atmosphere is selected from nitrogen, argon, and helium.

In some embodiments, the biomass raw materials have a concentration of 20-40 g/L.

In some embodiments, the phenolic hydroxyl-functionalized COFs have an amount of 0.05-0.5 g.

In some embodiments, the solvent has a volume of 30-50 mL.

The present invention is described in further detail below in connection with specific embodiments which should be noted as an interpretation of the present invention and not as a limitation.

### Example 1

A method for catalytic preparation of furfural from D-xylose using phenolic hydroxyl-functionalized covalent organic framework materials was provided in this example.

### (1) Preparation of Materials

During the preparation process of phenolic hydroxyl-functionalized COF materials, the raw material components were prepared in the following proportions: 170.13 mg of 1,3,5-tri-(4-aminophenyl) triazine, 119.61 mg of 2,5-dihydroxyterephthalaldehyde, 3 mL of n-butanol, 3 mL of o-dichlorobenzene, and 0.6 mL of acetic acid.

For the catalytic production of furfural using phenolic hydroxyl-functionalized COF materials, the raw material components were prepared in the following proportions: 1.2 g of D-xylose, 30 mL of solvent, and a certain amount of COF material.

### (2) Preparation of Phenolic Hydroxyl-Functionalized COF Material

The previously prepared 1,3,5-tri-(4-aminophenyl) triazine, n-butanol, 2,5-dihydroxyterephthalaldehyde, o-dichlorobenzene, and acetic acid were sequentially added to a thick-walled pressure-resistant tube and mixed uniformly. Following the solvothermal method, the reaction was carried out at 120° C. for 72 hours. After cooling, the product was washed three times with n-butanol, o-dichlorobenzene, and hot water, respectively, and then dried for 12 hours to obtain COF-A material.

(3) Application of Phenolic Hydroxyl-Functionalized COF Material in the Catalytic Preparation of Furfural from D-Xylose

The previously prepared D-xylose, solvent, and COF material were sequentially added to the reactor. The air inside the reactor was replaced with nitrogen gas 3-4 times, and the stirring rate was set to 500 rpm. The choice of solvent, reaction temperature, reaction time, and the amount of catalyst were shown in Table 1. After the reaction was completed, the reactor was cooled to room temperature, the liquid product was collected, and qualitative and quantitative analyses of the product were conducted. The results were shown in Table 1.

TABLE 1
Data table of different reaction conditions and results in Example 1

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water | 190 | 1 | 0.1 | 88.65 | 42.06 | 47.45 |
| 2 | Water | 200 | 1 | 0.1 | 88.70 | 56.56 | 63.77 |
| 3 | Water | 210 | 1 | 0.1 | 98.28 | 49.98 | 50.85 |
| 4 | Water | 200 | 0.5 | 0.1 | 84.40 | 47.08 | 55.78 |
| 5 | Water | 200 | 1 | 0.1 | 88.70 | 56.56 | 63.77 |
| 6 | Water | 200 | 1.5 | 0.1 | 93.56 | 50.56 | 54.04 |
| 7 | Water | 200 | 1 | 0.05 | 82.95 | 51.16 | 61.68 |
| 8 | Water | 200 | 1 | 0.1 | 88.70 | 56.56 | 63.77 |
| 9 | Water | 200 | 1 | 0.15 | 90.05 | 50.36 | 55.92 |
| 10 | Water- | 200 | 1 | 0.1 | 90.25 | 89.08 | 98.70 |
| | Tetrahydrofuran | | | | | | |
| 11 | Water-Toluene | 200 | 1 | 0.1 | 87.49 | 83.16 | 95.05 |
| 12 | Water-γ- | 200 | 1 | 0.1 | 90.80 | 73.55 | 81.00 |
| | Valerolactone | | | | | | |

The reference mark (^{※}) indicated that in the biphasic system composed of water and an organic solvent, the ratio of water to organic solvent was uniformly 1:1.

### Example 2

A method for catalytic preparation of furfural from xylan using phenolic hydroxyl-functionalized covalent organic framework materials was provided in this example.

### (1) Preparation of Materials

During the preparation process of phenolic hydroxyl-functionalized COF materials, the raw material components were prepared in the following proportions: 170.13 mg of 1,3,5-tri-(4-aminophenyl) triazine, 119.61 mg of 2,5-dihydroxyterephthalaldehyde, 3 mL of mesitylene, 3 mL of 1,4-dioxane, and 0.6 mL of acetic acid.

For the catalytic production of furfural using phenolic hydroxyl-functionalized COF materials, the raw material components were prepared in the following proportions: 1.2 g of xylan, 30 mL of solvent, and a certain amount of COF material.

### (2) Preparation of Phenolic Hydroxyl-Functionalized COF Material

The previously prepared 1,3,5-tri-(4-aminophenyl) triazine, mesitylene, 2,5-dihydroxyterephthalaldehyde, 1,4-dioxane and acetic acid were sequentially added to a thick-walled pressure-resistant tube and mixed uniformly. Following the solvothermal method, the reaction was carried out at 120° C. for 72 hours. After cooling, the product was washed three times with mesitylene, 1,4-dioxane, and hot water, respectively, and then dried for 12 hours to obtain COF-B material.

(3) Application of Phenolic Hydroxyl-Functionalized COF Material in the Catalytic Preparation of Furfural from Xylan

The previously prepared xylan, solvent, and COF material were sequentially added to the reactor. The air inside the reactor was replaced with argon gas 3-4 times, and the stirring rate was set to 500 rpm. The choice of solvent, reaction temperature, reaction time, and the amount of catalyst were shown in Table 2. After the reaction was completed, the reactor was cooled to room temperature, the liquid product was collected, and qualitative and quantitative analyses of the product were conducted. The results were shown in Table 2.

TABLE 2
Data table of different reaction conditions and results in Example 2

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water | 190 | 2 | 0.2 | 83.59 | 35.84 | 42.88 |
| 2 | Water | 200 | 2 | 0.2 | 83.77 | 48.35 | $7.72 |
| 3 | Water | 210 | 2 | 0.2 | 92.91 | 39.16 | 42.15 |
| 4 | Water | 200 | 1 | 0.2 | 81.01 | 41.02 | 50.64 |
| 5 | Water | 200 | 2 | 0.2 | 83.77 | 48.35 | 57.72 |
| 6 | Water | 200 | 3 | 0.2 | 87.39 | 41.36 | 47.33 |
| 7 | Water | 200 | 2 | 0.1 | 80.97 | 42.16 | 52.07 |
| 8 | Water | 200 | 2 | 0.2 | 83.77 | 48.35 | 57.72 |
| 9 | Water | 200 | 2 | 0.3 | 80.42 | 42.55 | 52.91 |
| 10 | Water- | 200 | 2 | 0.2 | 84.70 | 76.02 | 89.75 |
| | Tetrahydrofuran | | | | | | |
| 11 | Water-Toluene | 200 | 2 | 0.2 | 83.35 | 71.06 | 85.25 |
| 12 | Water-γ- | 200 | 2 | 0.2 | 85.86 | 61.59 | 71.73 |
| | Valerolactone | | | | | | |

The reference mark (^{※}) indicated that in the biphasic system composed of water and an organic solvent, the ratio of water to organic solvent was uniformly 1:1.

### Example 3

A method for catalytic preparation of furfural from arabinose using phenolic hydroxyl-functionalized covalent organic framework materials was provided in this example.

### (1) Preparation of Materials

During the preparation process of phenolic hydroxyl-functionalized COF materials, the raw material components were prepared in the following proportions: 170.13 mg of 1,3,5-tri-(4-aminophenyl) triazine, 119.61 mg of 2,5-dihydroxyterephthalaldehyde, 0.06 mg of scandium trifluoromethanesulfonate, 3 mL of mesitylene, 12 mL of 1,4-dioxane, and 150 mL of methanol.

For the catalytic production of furfural using phenolic hydroxyl-functionalized COF materials, the raw material components were prepared in the following proportions: 1.2 g of arabinose, 30 mL of solvent, and a certain amount of COF material.

### (2) Preparation of Phenolic Hydroxyl-Functionalized COF Material

The previously prepared 1,3,5-tri-(4-aminophenyl) triazine, 2,5-dihydroxyterephthalaldehyde, scandium trifluoromethanesulfonate, mesitylene, and 1,4-dioxane were sequentially added to a glass bottle, mixed evenly, and then left to stand for 30 minutes. Then, the mixture was subjected to a Soxhlet extraction with methanol for 12 hours. After cooling, the product was washed three times with mesitylene, 1,4-dioxane, and hot water, respectively. After drying for 12 hours, the COF-C material was obtained.

(3) Application of Phenolic Hydroxyl-Functionalized COF Material in the Catalytic Preparation of Furfural from Arabinose

The previously prepared arabinose, solvent, and COF material were sequentially added to the reactor. The air inside the reactor was replaced with helium gas 3-4 times, and the stirring rate was set to 500 rpm. The choice of solvent, reaction temperature, reaction time, and the amount of catalyst were shown in Table 3. After the reaction was completed, the reactor was cooled to room temperature, the liquid product was collected, and qualitative and quantitative analyses of the product were conducted. The results were shown in Table 3.

TABLE 3
Data table of different reaction conditions and results in Example 3

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water | 190 | 1 | 0.1 | 87.34 | 36.33 | 41.60 |
| 2 | Water | 200 | 1 | 0.1 | 88.30 | 46.22 | 52.35 |
| 3 | Water | 210 | 1 | 0.1 | 99.01 | 45.01 | 45.46 |
| 4 | Water | 200 | 0.5 | 0.1 | 84.01 | 39.41 | 46.91 |
| 5 | Water | 200 | 1 | 0.1 | 88.30 | 46.22 | 52.35 |
| 6 | Water | 200 | 1.5 | 0.1 | 94.98 | 46.50 | 48.96 |
| 7 | Water | 200 | 1 | 0.05 | 85.03 | 47.75 | 56.15 |
| 8 | Water | 200 | 1 | 0.1 | 88.30 | 46.22 | 52.35 |
| 9 | Water | 200 | 1 | 0.15 | 90.79 | 44.42 | 48.93 |
| 10 | Water- | 200 | 1 | 0.1 | 89.18 | 78.50 | 88.02 |
| | Tetrahydrofuran | | | | | | |
| 11 | Water-Toluene | 200 | 1 | 0.1 | 87.4 | 72.98 | 83.50 |
| 12 | Water-γ- | 200 | 1 | 0.1 | 91.00 | 74.89 | 82.30 |
| | Valerolactone | | | | | | |

The reference mark (^{※}) indicated that in the biphasic system composed of water and an organic solvent, the ratio of water to organic solvent was uniformly 1:1.

### Example 4

A method for catalytic preparation of furfural from xylan using phenolic hydroxyl-functionalized covalent organic framework materials was provided in this example.

### (1) Preparation of Materials

During the preparation process of phenolic hydroxyl-functionalized COF materials, the raw material components were prepared in the following proportions: 159.60 mg of 1,3,5-tri-(4-aminophenyl) triazine, 80.40 mg of 2-hydroxy-1,3,5-benzenetricarboxaldehyde, 9 mL of mesitylene, 9 mL of 1,4-dioxane, and 1.8 mL of acetic acid.

For the catalytic production of furfural using phenolic hydroxyl-functionalized COF materials, the raw material components were prepared in the following proportions: 1.2 g of D-xylose, 30 mL of solvent, and a certain amount of COF material.

### (2) Preparation of Phenolic Hydroxyl-Functionalized COF Material

The previously prepared 1,3,5-tri-(4-aminophenyl) triazine, mesitylene, 2-hydroxy-1,3,5-benzenetricarboxaldehyde, and 1,4-dioxane were sequentially added to a thick-walled pressure-resistant tube and mixed uniformly. Following the solvothermal method, the reaction was carried out at 120° C. for 72 hours. After cooling, the product was washed three times with mesitylene, 1,4-dioxane, and hot water, respectively, and then dried for 12 hours to obtain COF-D material.

(3) Application of Phenolic Hydroxyl-Functionalized COF Material in the Catalytic Preparation of Furfural from D-Xylose

The previously prepared D-xylose, solvent, and COF material were sequentially added to the reactor. The air inside the reactor was replaced with nitrogen gas 3-4 times, and the stirring rate was set to 500 rpm. The choice of reaction temperature, reaction time, and the amount of catalyst were shown in Table 4. After the reaction was completed, the reactor was cooled to room temperature, the liquid product was collected, and qualitative and quantitative analyses of the product were conducted. The results were shown in Table 4.

TABLE 4
Data table of different reaction conditions and results in Example 4

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water- | 170 | 2 | 0.2 | 86.38 | 70.26 | 81.34 |
| | Tetrahydrofuran | | | | | | |
| 2 | Water- | 180 | 2 | 0.2 | 87.60 | 77.09 | 88.00 |
| | Tetrahydrofuran | | | | | | |
| 3 | Water- | 190 | 2 | 0.2 | 88.01 | 74.02 | 84.10 |
| | Tetrahydrofuran | | | | | | |
| 4 | Water- | 180 | 1 | 0.2 | 87.04 | 73.6 | 84.56 |
| | Tetrahydrofuran | | | | | | |
| 5 | Water- | 180 | 2 | 0.2 | 87.60 | 77.09 | 88.00 |
| | Tetrahydrofuran | | | | | | |
| 6 | Water- | 180 | 3 | 0.2 | 88.02 | 72.81 | 82.72 |
| | Tetrahydrofuran | | | | | | |
| 7 | Water- | 180 | 2 | 0.1 | 84.78 | 75.02 | 88.49 |
| | Tetrahydrofuran | | | | | | |
| 8 | Water- | 180 | 2 | 0.2 | 87.60 | 77.09 | 88.00 |
| | Tetrahydrofuran | | | | | | |
| 9 | Water- | 180 | 2 | 0.3 | 90.06 | 73.62 | 81.75 |
| | Tetrahydrofuran | | | | | | |

The reference mark (^{※}) indicated that in the biphasic system composed of water and an organic solvent, the ratio of water to organic solvent was uniformly 1:1.

### Example 5

A method for catalytic preparation of furfural from xylan using phenolic hydroxyl-functionalized covalent organic framework materials was provided in this example.

### (1) Preparation of Materials

During the preparation process of phenolic hydroxyl-functionalized COF materials, the raw material components were prepared in the following proportions: 159.60 mg of 1,3,5-tri-(4-aminophenyl) triazine, 94.50 mg of 2,4,6-trihydroxybenzene-1,3,5-tricarboxaldehyde, 9 mL of mesitylene, 9 mL of 1,4-dioxane, and 1.8 mL of acetic acid.

For the catalytic production of furfural using phenolic hydroxyl-functionalized COF materials, the raw material components were prepared in the following proportions: 1.2 g of D-xylose, 30 mL of solvent, and a certain amount of COF material.

### (2) Preparation of Phenolic Hydroxyl-Functionalized COF Material

The previously prepared 1,3,5-tri-(4-aminophenyl) triazine, mesitylene, 2,4,6-trihydroxybenzene-1,3,5-tricarboxaldehyde, and 1,4-dioxane were sequentially added to a thick-walled pressure-resistant tube and mixed uniformly. Following the solvothermal method, the reaction was carried out at 120° C. for 72 hours. After cooling, the product was washed three times with mesitylene, 1,4-dioxane, and hot water, respectively, and then dried for 12 hours to obtain COF-E material.

(3) Application of Phenolic Hydroxyl-Functionalized COF Material in the Catalytic Preparation of Furfural from D-Xylose

The previously prepared D-xylose, solvent, and COF material were sequentially added to the reactor. The air inside the reactor was replaced with nitrogen gas 3-4 times, and the stirring rate was set to 500 rpm. The choice of reaction temperature, reaction time, and the amount of catalyst were shown in Table 5. After the reaction was completed, the reactor was cooled to room temperature, the liquid product was collected, and qualitative and quantitative analyses of the product were conducted. The results were shown in Table 5.

TABLE 5
Data table of different reaction conditions and results in Example 5

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water- | 170 | 1 | 0.1 | 85.07 | 72.11 | 84.77 |
| | Tetrahydrofuran | | | | | | |
| 2 | Water- | 180 | 1 | 0.1 | 88.48 | 80.07 | 90.50 |
| | Tetrahydrofuran | | | | | | |
| 3 | Water- | 190 | 1 | 0.1 | 92.19 | 75.05 | 81.41 |
| | Tetrahydrofuran | | | | | | |
| 4 | Water- | 180 | 0.5 | 0.1 | 80.29 | 71.48 | 89.03 |
| | Tetrahydrofuran | | | | | | |
| 5 | Water- | 180 | 1 | 0.1 | 88.48 | 80.07 | 90.50 |
| | Tetrahydrofuran | | | | | | |
| 6 | Water- | 180 | 2 | 0.1 | 90.01 | 78.49 | 87.20 |
| | Tetrahydrofuran | | | | | | |
| 7 | Water- | 180 | 1 | 0.05 | 87.92 | 73.92 | 84.08 |
| | Tetrahydrofuran | | | | | | |
| 8 | Water- | 180 | 1 | 0.1 | 88.48 | 80.07 | 90.50 |
| | Tetrahydrofuran | | | | | | |
| 9 | Water- | 180 | 1 | 0.2 | 91.28 | 72.48 | 79.40 |
| | Tetrahydrofuran | | | | | | |

The reference mark (^{※}) indicated that in the biphasic system composed of water and an organic solvent, the ratio of water to organic solvent was uniformly 1:1.

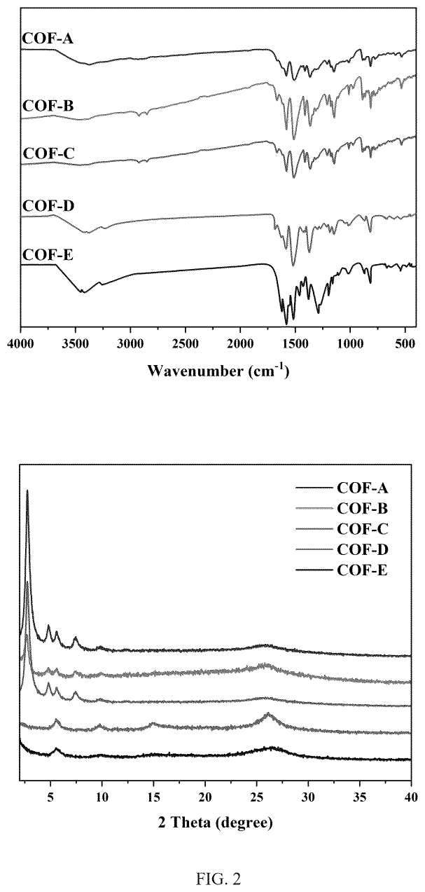

From FIG. 2, it can be seen that the phenolic hydroxyl-functionalized COFs material has been successfully prepared by the present invention.

As can be observed from Tables 1-5, the phenolic hydroxyl-functionalized covalent organic framework material exhibited high catalytic activity in the process of catalyzing the preparation of furfural from biomass, especially in a biphasic solvent system consisting of water and organic solvent. Furthermore, the yield of furfural initially increased and then decreased with the increase of reaction temperature, reaction time, and catalyst dosage. This is because excessively high temperatures, prolonged reaction times, and excessive amounts can all lead to the further decomposition of furfural, forming a variety of by-products and reducing the yield of furfural.

### Example 6

In the example, the reusability of the phenolic hydroxyl-functionalized COFs material described in Examples 1-5 was tested. The COF materials that were used for catalyzing the preparation of furfural from biomass in Examples 1-5 were filtered, recovered, washed, and then dried in an oven for 12 hours.

0.1 g of the COF material dried to constant weight was mixed with 1.2 g of D-xylose and 30 mL of a water-tetrahydrofuran solution, and then added to the reactor. The air inside the reactor was replaced with nitrogen gas 3-4 times, with the reaction temperature set to 200° C., reaction time to 1 hour, and stirring rate to 500 rpm. After the reaction was completed, the reactor was cooled to room temperature, the liquid product was collected, and qualitative and quantitative analyses of the product were conducted. This process was repeated 3 times. The results were shown in Table 6.

TABLE 6
Data table of different reaction conditions and results in Example 6

| | | | | | | Furfural | |
|---|---|---|---|---|---|---|---|
| | | Temperature, | Time, | Catalyst, | Conversion | Yield, | Selectivity, |
| No. | Solvent^{※} | ° C. | h | g | Rate, % | % | % |
| 1 | Water- | 200 | 1 | 0.1 | 90.25 | 89.08 | 98.70 |
| | Tetrahydrofuran | | | | | | |
| 2 | Water- | 200 | 1 | 0.1 | 89.93 | 88.06 | 97.92 |
| | Tetrahydrofuran | | | | | | |
| 3 | Water- | 200 | 1 | 0.1 | 89.02 | 87.74 | 98.56 |
| | Tetrahydrofuran | | | | | | |
| 4 | Water- | 200 | 1 | 0.1 | 84.78 | 74.72 | 88.13 |
| | Tetrahydrofuran | | | | | | |
| 5 | Water- | 200 | 1 | 0.1 | 85.02 | 73.97 | 87.00 |
| | Tetrahydrofuran | | | | | | |
| 6 | Water- | 200 | 1 | 0.1 | 84.93 | 73.15 | 86.13 |
| | Tetrahydrofuran | | | | | | |
| 7 | Water- | 200 | 1 | 0.1 | 90.14 | 79.25 | 87.92 |
| | Tetrahydrofuran | | | | | | |
| 8 | Water- | 200 | 1 | 0.1 | 88.02 | 79.25 | 90.04 |
| | Tetrahydrofuran | | | | | | |
| 9 | Water- | 200 | 1 | 0.1 | 89.28 | 78.98 | 88.46 |
| | Tetrahydrofuran | | | | | | |
| 10 | Water- | 200 | 1 | 0.1 | 88.35 | 67.01 | 75.85 |
| | Tetrahydrofuran | | | | | | |
| 11 | Water- | 200 | 1 | 0.1 | 88.00 | 67.23 | 76.40 |
| | Tetrahydrofuran | | | | | | |
| 12 | Water- | 200 | 1 | 0.1 | 86.21 | 66.98 | 77.69 |
| | Tetrahydrofuran | | | | | | |
| 13 | Water- | 200 | 1 | 0.1 | 91.95 | 76.41 | 83.10 |
| | Tetrahydrofuran | | | | | | |
| 14 | Water- | 200 | 1 | 0.1 | 90.91 | 76.12 | 83.73 |
| | Tetrahydrofuran | | | | | | |

The reference mark (^{※}) indicated that in the biphasic system composed of water and an organic solvent, the ratio of water to organic solvent was uniformly 1:1.
Note:
Nos. 1-3 were the reusability experiments for COF-A material in Example 1; Nos. 4-6 were for COF-B material in Example 2; Nos. 7-9 were for COF-C material in Example 3; Nos. 10-12 were for COF-D material in Example 4; Nos. 13-15 were for COF-E material in Example 5.

Table 6 demonstrated that the phenolic hydroxyl-functionalized COFs material exhibited excellent recyclability and reusability in the catalytic preparation of furfural from biomass. After being used three times, there were no significant changes in the degradation rate of xylose, the yield of furfural, or the selectivity for furfural.

The above descriptions are merely preferred embodiments of the present invention and are not intended to limit the present invention. For those skilled in the art, various changes and modifications can be made to the present invention. Any modification, equivalent replacement, or improvement made within the spirit and principles of the present invention should be included within the scope of the present invention's protection.

What is claimed is:

1. A method for catalytic preparation of furfural from biomass using phenolic hydroxyl-functionalized covalent organic framework materials, wherein comprising:
using aldehyde or/and amino compounds containing phenolic hydroxyl functional groups as monomers, through solvothermal, microwave, or grinding methods, to initiate a Schiff base reaction followed by dehydration condensation to synthesize phenolic hydroxyl-functionalized covalent organic frameworks (COFs); and
mixing biomass raw materials and the phenolic hydroxyl-functionalized COFs in a solvent to achieve a uniform mixture, which is then subjected to catalytic reaction under an inert atmosphere at 160-210° C. for 0.5-5 hours to produce furfural.

2. The method according to claim 1, wherein the biomass raw materials comprise xylan, xylose, arabinose, and biomass hydrolysate.

3. The method according to claim 1, wherein the solvent is at least one of water, tetrahydrofuran, toluene, and γ-valerolactone.

4. The method according to claim 1, wherein the inert atmosphere is selected from nitrogen, argon, and helium.

5. The method according to claim 1, wherein the biomass raw materials have a concentration of 20-40 g/L.

6. The method according to claim 1, wherein the phenolic hydroxyl-functionalized COFs have an amount of 0.05-0.5 g.

7. The method according to claim 1, wherein the solvent has a volume of 30-50 mL.

8. A method for preparation of phenolic hydroxyl-functionalized covalent organic framework materials, wherein:
using 1,3,5-tri-(4-aminophenyl) triazine and 2,5-dihydroxyterephthalaldehyde as monomers, through solvothermal, microwave, or grinding methods, to initiate a Schiff base reaction followed by dehydration condensation to synthesize phenol hydroxyl-functionalized covalent organic frameworks (COFs).

9. Phenolic hydroxyl-functionalized covalent organic frameworks materials prepared according to a method of claim 8.

10. An application of phenolic hydroxyl-functionalized covalent organic framework materials of claim 9 in the catalytic preparation of furfural from biomass.
